(19) United States
(12) Plant Patent Application Publication
(10) Pub. No.: US 2016/0205847 P1
(43) Pub. Date: Jul. 14, 2016

(54) Robinia pseudoacacia 'Huangjin' plant named 'Golden Robinia'

(50) ;

(71) Applicant: Henan Mingpin Colorized Seedling Co., Ltd, Suiping (CN)

(72) Inventor: Huaming Wang, Suiping (CN)

(21) Appl. No.: 14/999,004

(22) Filed: Mar. 16, 2016

(51) Int. Cl. A01H 5/00

(52) U.S. Cl. PLT/216

(57) ABSTRACT

‘Golden Robinia’ is a distinctive variety of Robinia Linn. Plant, which is characterized by that leaves are kept golden in spring, summer and autumn. The Golden Robinia was discovered by the applicant according to: 1) On Jul. 5, 2008, a five-year-old Golden Locust was mutagenized; and on Jul. 22, 2008, a mutant branch, whose leaves were kept golden, was found; then the mutant branch was grafted onto a Robinia rootstock. 2) In the end of August 2008, it was observed that sprouts of the grafted plant were still golden. 3) In spring of 2009, it was observed that leaves of the mother plant and the grafted planted were still golden; furthermore, the leaves were golden and never turned to green in summer and autumn. 4) In the following years, bud and branch grafting methods were used for multiplying, wherein characteristics thereof were kept still.

### LATIN NAME

Robinia pseudoacacia ‘Huangjin’

### VARIETY DENOMINATION

Golden Robinia

### BACKGROUND OF THE INVENTION

The present invention relates to a new and distinct variety of Robinia pseudoacacia ‘Huangjin’ plant, which is given the varietal denomination of ‘Golden Robinia’.

The new Golden Robinia was discovered by the applicant according to:

1) On Jul. 5, 2008, a five-year-old Golden Locust was mutagenized; and on Jul. 22, 2008, a mutant branch, whose leaves were kept golden, was found; then the mutant branch was grafted onto a Robinia rootstock.

2) In the end of August 2008, it was observed that sprouts of the grafted plant were still golden.

3) In spring of 2009, it was observed that leaves of the mother plant and the grafted planted were still golden; furthermore, the leaves were golden and never turned to green in summer and autumn.

4) In the following years, bud and branch grafting methods were used for multiplying, wherein characteristics thereof were kept still.

### SUMMARY OF THE NEW VARIETY

‘Golden Robinia’ is a distinctive variety of Robinia pseudoacacia ‘Huangjin’ plant. The Golden Robinia is a broadleaf deciduous arbor, wherein a crown thereof is umbrella-shaped and dense; a trunk thereof has middle-depth cracks; bark thereof is dark brown bark; branches thereof are straight and bias splays with stipule thorns thereon. The branches in a year of birth are brown without indumentum or are puberulent when being young. Lateral branches have middle thicknesses, and odd-pinnate compound leaves alternately grow thereon, wherein there are 9-19 leaflets, petioles are 1-3 cm long, and petiolules are about 2 cm long and pubescent. The compound leaves have middle lengths. The leaflets have middle sizes, wherein the leaflets are ovate or ovate-oblong with a length of 2.5-5 cm and a width of 1.5-3 cm, wherein bases thereof are wide-cuneate or round, and apexes thereof are round or slightly concave with small thorns. The leaves are integrifolious and golden without stipule thorns.

Racemes are axillary and shorter than the leaves, and rachises are brown and sparsely haired. Pedicels are 8-13 mm long and pubescent, wherein the pedicels are calyx-bell-shaped with 5-toothed irregular cracks, and surfaces thereof are pubescent. Corollas are white and fragrant, and standards are suborbicular with lengths of 18 mm, wherein bases thereof are clawed, and apexes thereof are slightly concave. Wings are obovate, wherein bases thereof have slender claws, and tops thereof are round and 18 mm long. Keels are curved inwards, wherein bases thereof have long claws. There are 10 stamens, which are divided into 9 and 1. Ovaries are linear-oblong with white short hairs, and stylus are bent at almost right angles.

Pods are flat, linear-oblong, 3-11 cm long, brown, and smooth. Each of the pods comprises 3-10 seeds, and two splits. According to the Golden Robinia, a period of flowers and fruits is from May to September.

A most distinctive feature of the Golden Robinia is that the leaves are kept golden in spring, summer and autumn, and will not change. As a contrast, a leaf color of Golden Locust changes with seasons, wherein the leaf color of the Golden Locust is golden in spring, yellow-green in summer, and orange in autumn; which means the leaf color is varied. Additionally, a growth potential of the Golden Robinia is weaker than that of the Golden Locust.

### BRIEF DESCRIPTION OF THE DRAWINGS

FIG. 1 is a color photograph of the new invention, ‘Golden Robinia’, showing whole plants.

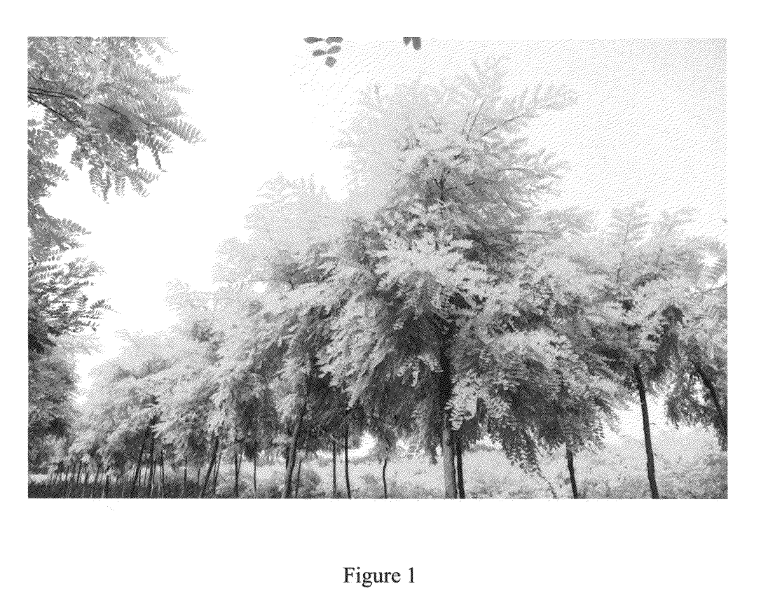

FIG. 2 is a color photograph of the new invention, ‘Golden Robinia’, showing a mother plant.

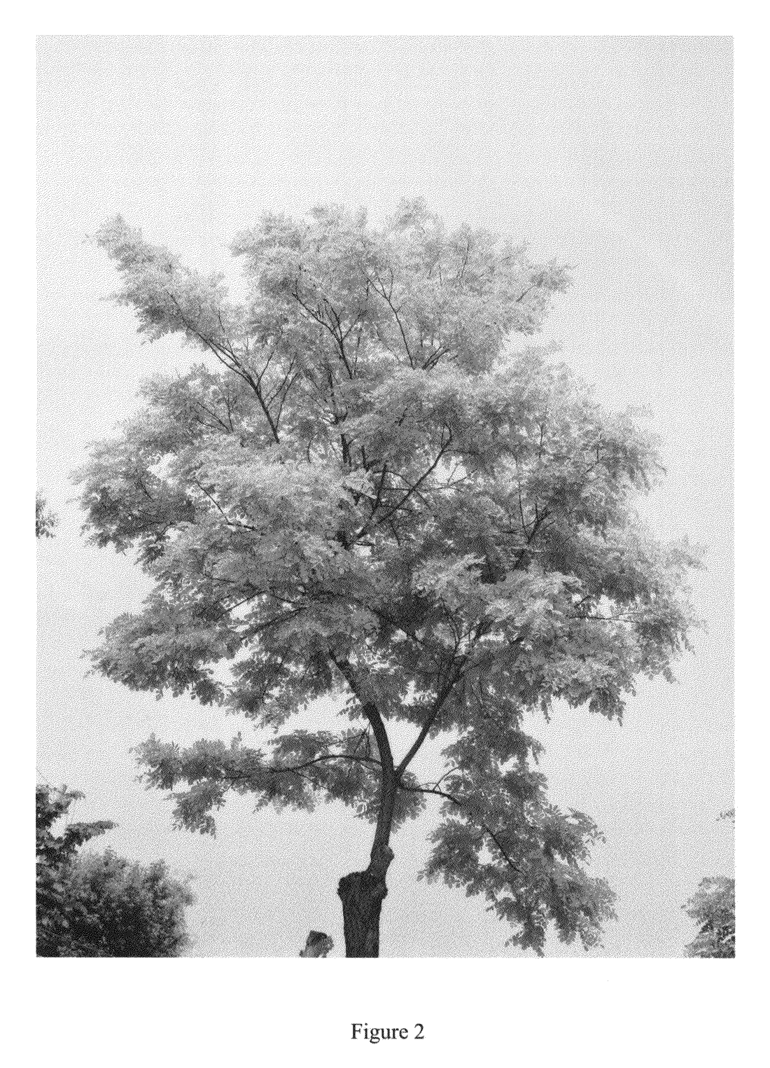

FIG. 3 is a color photograph of the new invention, ‘Golden Robinia’, showing spring leaves.

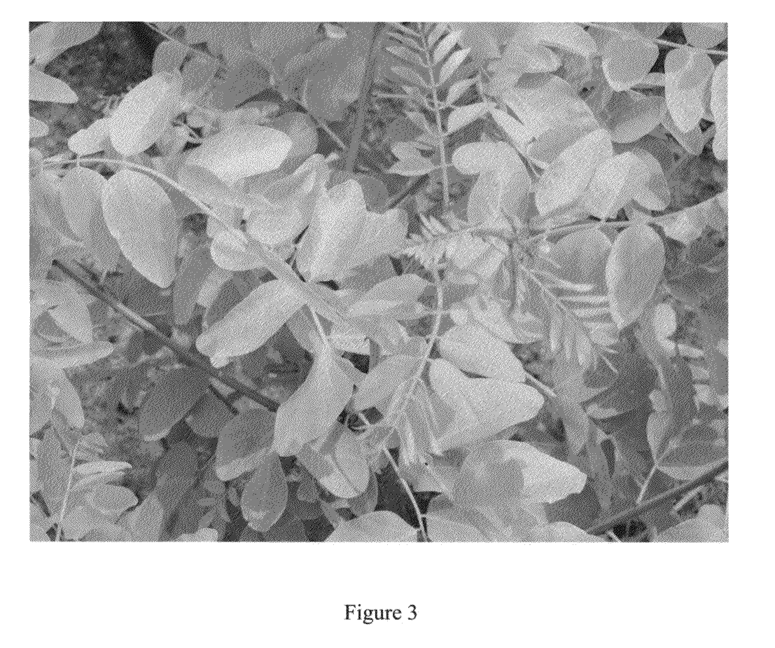

FIG. 4 is a color photograph of the new invention, ‘Golden Robinia’, showing summer leaves.

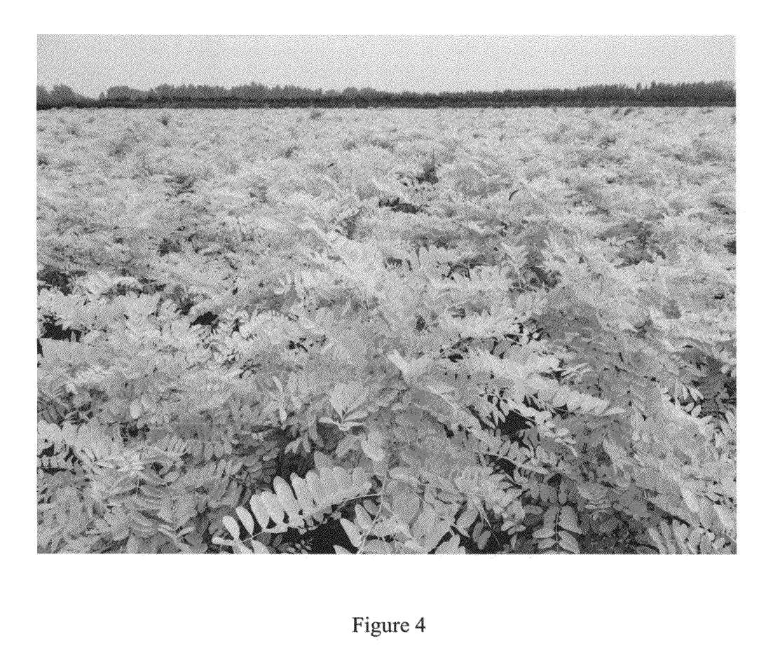

FIG. 5 is a color photograph of the new invention, ‘Golden Robinia’, showing autumn leaves.

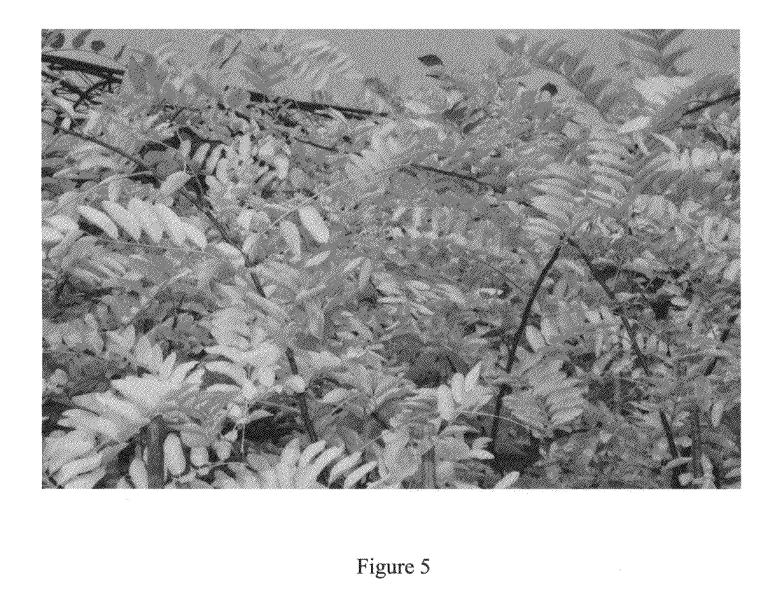

FIG. 6 is a color photograph of the new invention, ‘Golden Robinia’, showing comparison of the Golden Robinia and Golden Locust.

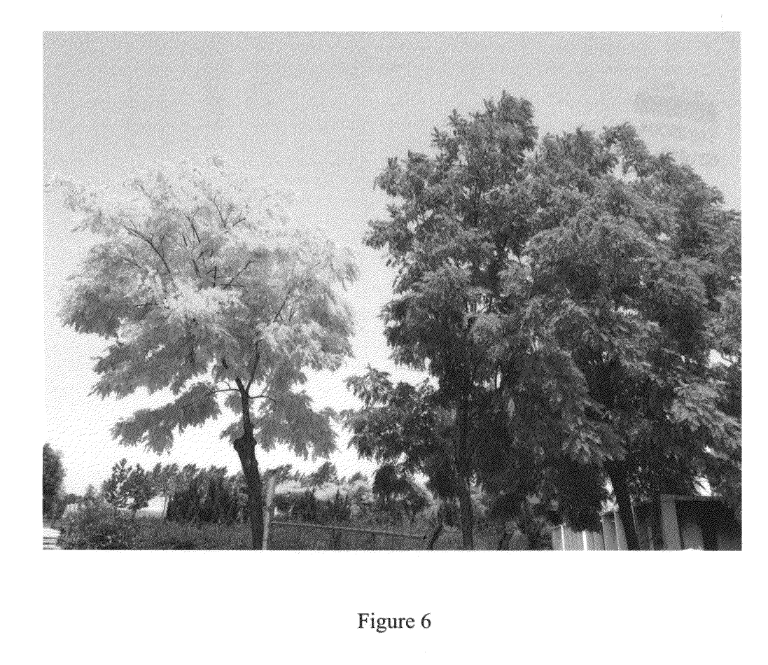

### DETAILED DESCRIPTION

The accompanying colored photographs illustrate the overall appearance of the new and distinct variety of the Robinia pseudoacacia ‘Huangjin’ plant, which is known as ‘Golden Robinia’. The descriptions disclosed herein are based upon observations of the plant growth.

The Golden Robinia is a broadleaf deciduous arbor, wherein a crown thereof is umbrella-shaped and dense; a trunk thereof has middle-depth cracks; bark thereof is dark brown bark; branches thereof are straight and bias splays with stipule thorns thereon. The branches in a year of birth are brown. Odd-pinnate compound leaves alternately grow. Leaflets are ovate or ovate-oblong. Leaves are integrifolious and golden without stipule thorns. Racemes are axillary. Corollas are white and fragrant. Pods are flat, linear-oblong, brown, and smooth. Each of the pods comprises 3-10 seeds, and two splits.

A main distinctive feature of the Golden Robinia is that the leaves are kept golden in spring, summer and autumn. Additionally, a growth potential of the Golden Robinia is weaker than that of the Golden Locust.

| | feature | | | |
|---|---|---|---|---|
| | leaf color | | | |
| name | spring | summer | autumn | growth potential |
| Golden Robinia | golden | golden | golden | slightly weak |
| Golden Locust | golden | green | orange | strong |

By years of asexual reproduction, the Golden Robinia is consistent and stable.

The Golden Robinia has a strong adaptability, which is able to grow at any place which is suitable for Robinia to grow. For example, the Golden Robinia is able to grow within an area covering north latitude 23°-46°, and east longitude 124°-86°. A highest allowable altitude is up to 2100 m.

The Golden Robinia demands light. A warm and humid climate is preferred, wherein the Golden Robinia grows well at areas with an annual average temperature of 8-14° C., and an annual precipitation of 500-900 mm. The Golden Robinia has a low requirement for soil due to a strong adaptability. However, thick, fertile, loose and moist powder sands, sandy soil and loam soil are preferred. The Golden Robinia does not sensitive to soil pH values. On subsoil that is too heavy and hard, poorly drained clay, or coarse sands, the Golden Robinia grows poorly. Although there is a certain drought resistance, the Golden Robinia always suffers die-back during severe drought seasons without rain. The Golden Robinia has a poor wet intolerance and is afraid of wind. The Golden Robinia belongs to a fast-growing type with shallow roots, dense crowns, early fruits, and an abundant yield.

These features and other characteristics of the plant are apparent from the figures.

What is claimed is:

1. A new and distinct variety of plant named ‘Golden Robinia’, substantially as described and illustrated, classified in the family Robinia Linn., and characterized by that leaves are kept golden in spring, summer and autumn.
